(19) United States
(12) Patent Application Publication
(10) Pub. No.: US 2016/0000844 A1
(43) Pub. Date: Jan. 7, 2016

(54) A COMPOSITION FOR IMPROVING JOINT HEALTH, OR FOR PREVENTING AND TREATING RHEUMATOID ARTHRITIS AND OSTEOARTHRITIS

(71) Applicant: HANPOONG PHARM & FOODS CO., LTD., Jeonju-si (KR)

(72) Inventors: Hyoung-kwon CHO, Jeonju-si (KR); Ki-young JUN, Jeonju-si (KR); Jun-kyoung LEE, Imsil-gun (KR); Chang-hyun JEON, Wanju-gun (KR); Dae-sung KIM, Iksan-si (KR); Erisa BYUN, Jeonju-si (KR); Jin-ju YU, Wanju-gun (KR); Young-bae KWON, Jeonju-si (KR); Jung-kee KWON, Damyang-gun (KR); Dae-ki KIM, Iksan-si (KR); Se-gun KIM, Donghae-si (KR); Hyeon-ju JEONG, Iksan-si (KR); Eun-joo LEE, Daejeon (KR)

(21) Appl. No.: 14/769,824

(22) PCT Filed: Dec. 13, 2013
(86) PCT No.: PCT/KR2013/011598

§ 371 Date: Aug. 24, 2015

(30) Foreign Application Priority Data
Mar. 29, 2013 (KR) 10-2013-0034232

(51) Int. Cl. A61K 36/25; A61K 36/232; A23L 1/30; A61K 36/78; A61K 36/9068; A61K 36/732; A61K 36/31

(52) U.S. Cl. CPC A61K 36/25; A61K 36/732; A61K 36/232; A61K 36/31; A61K 36/78; A61K 36/9068; A23L 1/3002; A61K 2236/39; A23V 2002/00

(57) ABSTRACT

The present invention relates to a composition for helping with joint health or preventing, alleviating or treating rheumatoid arthritis and osteoarthritis, which is based on the results of tests conducted using an inflammatory cell model, an osteoarthritis animal model and an acute arthritis animal model.

### TECHNICAL FIELD

The present invention relates to a composition for helping with joint health or preventing, alleviating or treating rheumatoid arthritis and osteoarthritis.

In particular, the present invention relates to a composition for helping with joint health or preventing, alleviating or treating rheumatoid arthritis and osteoarthritis, which has effects on the inhibition of inflammatory cytokines and matrix metalloproteinases (MMPs), the relief of inflammation and pain, the enhancement of activity of cartilage formation factors, and the normalization of the joint structure.

Furthermore, the present invention relates to a composition for helping with joint health or preventing, alleviating or treating rheumatoid arthritis and osteoarthritis, which has the above effects and is also not cytotoxic to cartilage tissue and synovial tissue, thereby enabling the joint structure to be protected and maintained.

Moreover, the present invention relates to a composition for helping with joint health or preventing, alleviating or treating rheumatoid arthritis and osteoarthritis, which is based on the results of tests conducted using an inflammatory cell model, an osteoarthritis animal model and an acute arthritis animal model.

### BACKGROUND ART

Arthritis is a disease that causes inflammation and pain, and can be chiefly divided into osteoarthritis (degenerative arthritis) and rheumatoid arthritis.

Osteoarthritis, also called degenerative arthritis, occurs mainly in the elderly without a specific organic cause, and will cause movement disorders, such as gait disturbances, due to the deformation of the joint structure when it progresses to a chronic state.

Osteoarthritis causes damage to the bond and ligament of the joint mainly due to progressive damage to joint cartilage or degenerative changes in joint cartilage, thereby causing inflammation and pain.

The mechanism of osteoarthritis is as follows. As osteoarthritis progresses, the production of inflammatory cytokines such as TNF-α, IL-1 and the like increases, the secretion of matrix metalloproteinases (MMPs), such as collagenase, stromelysin and the like, increases, thereby causing the breakdown of joint cartilage. MMPs induce IL-1, TNF-α and the like, which affect tissues, such as muscle, tendon, ligament and the like, resulting in severe pain.

In this case, MMPs (matrix metalloproteinases) are major factors that are involved in cartilage damage, and can be divided into collagenases, gelatinases, stromelysins, and membrane-type MMPs and several other groups of MMPs.

Rheumatoid arthritis is an inflammatory disease characterized by polyarthritis, and autoimmunity is known as the major cause of rheumatoid arthritis.

The symptoms of rheumatoid arthritis are as follows. As inflammation occurs in synovial membrane tissue, macrophages, dendritic cells, T lymphocytes, B lymphocytes and the like migrate to synovial tissue, and as a result, the amount of joint fluid increases so that the joint swells to cause pain.

When the hyperplasia of inflammatory synovial tissue occurs while such inflammation continues, the bone and cartilage are destroyed, and as a result, the joint structure is deformed and movement disorders occur.

In addition, it is known that, in the case of rheumatoid arthritis, the production of inflammatory cytokines, such as TNF-α and IL-1, increases to increase the secretion of MMPs, thereby destroying collagen and proteoglycans and thus destroy joint cartilage.

Furthermore, as the progression of osteoarthritis or rheumatoid arthritis continues, changes in the joint structure, such as the destruction of joint cartilage, occur. Roughly speaking, the spacing between joints becomes progressively narrower, and sclerosis of articular cartilage occurs. In addition, when arthritis progresses, bone spurs are formed at the edge of the articular surface, and the articular surface becomes irregular. In summary, in the case of osteoarthritis and rheumatoid arthritis, inflammation and pain are caused by inflammatory cytokines and MMPs, and changes in the joint structure, such as cartilage destruction, occur. Accordingly, there is an urgent need for a drug that can inhibit inflammatory cytokines and matrix metalloproteinases (MMPs), relieve inflammation and pain, enhance the activity of cartilage formation factors, and normalize the joint structure.

Hereinafter, natural materials used in the present invention will be described in brief.

Kalopanacis Cortex is the bark of Kalopanax pictus Nakai, which is a deciduous tree belonging to the family Araliaceae, or the bark of Kalopanax pictus Nakai var. chinensis Nakai, Kalopanax pictus Nakai var. magnificus Nakai and Kalopanax pictus Nakai var. maximowiczii Nakai, which belong to the same family.

It is known that Kalopanacis Cortex has a tannin content of 13-30%, and contains glucose, kalotoxin, kalosaponin, liriodendrin, hederagenin, arabinose, benzoic acid, amino acid, d-mannitol and polyacetylene, and the main components thereof are saponin, phenolic glycosides, etc.

Kalopanacis cortex is widely used for neuralgia, joint pain, lumbago, syphilis, diarrhea, toothache, diabetes, tonic activity and the like in folk remedies.

Chaenomelis Fructus is the ripe fruit of Chaenomeles sinensis Koehne or Chaenmeles speciosa Nakai, which is a deciduous shrub belonging to the family Rosaceae.

Chaenomelis Fructus is known to contain saponin, malic acid, tartaric acid, citric acid, vitamin C, flavonoids, tannin, etc.

In Chinese medicine, Chaenomelis Fructus is recognized as a drug that harmonizes the spleen and stomach and removes dampness. Accordingly, it is used as an effective drug for acute gastrointestinal diseases, beriberi, muscle pain, arthritis and neuralgia. Furthermore, Chaenomelis Fructus is known to have antitussive and expectorant activities and to be effective against pneumonia, bronchitis and the like.

Angelicae Gigantis is the dried root of Angelica gigas Nakai that is a perennial plant belonging to the family Umbelliferae.

Angelicae Gigantis mainly contains butylidene phthalide, n-valerop-henone-o-carboxylic acid, delta-2,4-dihydrophthalic anhydride, a large amount (40%) of vitamin B12 (0.25-4 mg/100 g), and vitamin A (0.0675%). In addition, it contains isoimperatorin, umbelliferone, nodakenetin, angelinol, gigasol, prenyletin, nodakenin, decursinol, imperatorin, decursin, decursidin, decursinol angelate, choline, α-pinene, myrcene, ρ-cymene, etc.

Angelicae Gigantis has anticancer effects, blood pressure-lowering effects, the effect of increasing blood flow in coronary arteries, the effect of increasing leukocyte production, etc.

Raphani Semen is the seed of annual or biennial Raphanus sativus L. belonging to the family Cruciferae or other plants belonging to the same family.

Raphani Semen contains fatty oils and essential oils. The essential oils contain methylthiole and the like, and the fatty oils contain large amounts of erucic acid, linoleic acid, and an ester of glycerinsinapic acid.

Raphani Semen has the effects of allowing “qi” to flow and relieving indigestion, and thus is used mainly for abdominal dropsy, belching, gastric hyperacidity, diarrhea, etc. It is known to be effective in treating a loss of appetite, loosening old phlegm and easing chronic coughs.

Saururi Herba is the root and whole plant of Saururus chinensis Baill. that is a perennial orchid belonging to the family Saururaceae.

Saururi Herba contains quercetin, quercitrin, isoquercetin, quercetin-3-1-arabinoside, hiperin, rutin and the like, and the main component of an essential oil contained in the whole plant of Saururi Herba is methyl-n-nonyl ketrone. Saururi Herba has fever alleviation, irrigation and tumor removal effects. Thus, in Chinese medicine, the dried whole plant of Saururi Herba is used for body swelling, difficult urination, gonorrheal infection, cancers, beriberi, jaundice, hepatitis, etc.

Zingiberis Rhizoma Crudus is the rhizome of Zingiber officinale Roscoe that is a perennial plant belonging to the family Zingiberaceae.

Essential oil contained in Zingiberis Rhizoma Crudus contains α-zingiberene, β-santalol, ρ-phellandrene, β-bisabolene, α-curcumene, zingiberol, perillaldehyde, neral, geranial, 2-caraneol, camphene, β-ocimene, myrcene, citral, isofenchyl alchole, etc. In addition, it contains components having a hot taste, including gingerol, methylgingediol, methylgingediacetate, gingerdione, etc. In this case, gingerol is decomposed into a mixture of shogaol, zingerone and zingiberone.

Furthermore, Zingiberis Rhizoma Crudus contains furanogermenone, several kinds of amino acids, including pipecolic acid, aspartic acid, glutamic acid and serine, as well as resinous materials and starch. The applicant has developed a composition for preventing, alleviating or treating rheumatoid arthritis and osteoarthritis, which is prepared using Kalopanacis Cortex, Chaenomelis Fructus, Angelicae Gigantis, Raphani Semen, Saururi Herba, and zingiberis rhizome crudus as described above. Prior arts related to these materials are described in brief below.

First, Korean Patent Application Publication No. 10-2011-0016825 discloses a composition for preventing or treating arthritis, which contains a mixed ingredient of Schizandra chinensis, Scutellaria root and Kalopanacis Cortex as an active ingredient. Furthermore, Korean Patent Application Publication No. 10-2003-0091405 discloses a composition for preventing or treating arthritis, which contains an ethyl acetate soluble extract of Kalopanacis Cortex as an active ingredient.

Furthermore, Korean Patent Application Publication No. 10-2011-0038631 discloses a composition for cartilage regeneration, pain suppression and edema suppression, which contains, as an active ingredient, extracts of Chaenomelis Fructus, Achyranthis radix, Acanthopanax senticosus, Cinnamomum cassia bark, Gentianae macrophyllae radix, Clematis chinensis root, Angelica root, Cnidium officinale root, Gastrodia elata, Carthamus tinctorius, Dipsasi radix and Saposhnikovia divaricata root.

Furthermore, Korean Patent Application Publication No. 2002-0011221 discloses an herbal medicinal composition, which is prepared from Saururi Herba and Houttuynia Cordata and has an anticancer effect. In addition, Korean Patent Application Publication No. 10-2004-0063673 discloses an herbal medicinal composition for treating arthritis, which is prepared using wild peach, Zingiberis Rhizoma Crudus, onion, Cinnamomi Ramulus, Ganoderma lucidum, Kalopanax pictus NAKAI and edible porcine bone meal.

In summary, although it can be seen that Kalopanacis Cortex, Chaenomelis Fructus, Angelicae Gigantis, Saururi Herba and Zingiberis Rhizoma Crudus are used in the prior arts related to arthritis, they are used chiefly by mixing them with other materials and extracting the mixture. In addition, for Raphani Semen, a prior art related to arthritis has not been found.

### DISCLOSURE

### Technical Problem

The present invention is intended to provide a composition for helping with joint health or preventing, alleviating or treating rheumatoid arthritis and osteoarthritis, which has effects on the inhibition of inflammatory cytokines and matrix metalloproteinases (MMPs), the relief of inflammation and pain, the enhancement of activity of cartilage formation factors, and the normalization of the joint structure.

The present invention is also intended to provide a composition for helping with joint health or preventing, alleviating or treating rheumatoid arthritis and osteoarthritis, which has the above effects and which is also not cytotoxic to cartilage tissue and synovial tissue, thereby enabling the joint structure to be protected and maintained.

The present invention is also intended to provide a composition for helping with joint health or preventing, alleviating or treating rheumatoid arthritis and osteoarthritis, which is based on the results of tests conducted using an inflammatory cell model, an osteoarthritis animal model and an acute arthritis animal model.

### Technical Solution

In order to accomplish the above objects, the present invention provides a composition for helping with joint health or preventing, alleviating or treating rheumatoid arthritis and osteoarthritis, which contains, as an active ingredient:

an extract mixture obtained by extracting each of Kalopanacis Cortex, Chaenomelis Fructus, Angelicae Gigantis, Zingiberis Rhizoma Crudus, Raphani Semen and Saururi Herba to prepare extracts, and mixing the extracts with one another at a mixing ratio of 150-250 parts by weight of the Kalopanacis Cortex extract: 650-750 parts by weight of the Chaenomelis Fructus extract: 650-750 parts by weight of the Angelicae Gigantis extract: 50-150 parts by weight of the Zingiberis Rhizoma Crudus extract: 50-150 parts by weight of the Raphani Semen extract: 150-250 parts by weight of the Saururi Herba extract; or

a dry extract mixture obtained by drying each of the extracts and mixing the dried extracts with one another at the above mixing ratio; or

a mixed extract obtained by mixing Kalopanacis Cortex, Chaenomelis Fructus, Angelicae Gigantis, Zingiberis Rhizoma Crudus, Raphani Semen and Saururi Herba with one another at the above mixing ratio to prepare a mixture, and extracting the mixture.

In the present invention, the composition may have effects on the inhibition of inflammatory cytokines and matrix metalloproteinases (MMPs), the relief of inflammation and pain, the enhancement of activity of cartilage formation factors, and the normalization of the joint structure.

In the present invention, the number of times the extraction is performed when each of the extracts or mixed extract is prepared may be two or more.

The present invention may also provide a pharmaceutical composition for alleviating or treating rheumatoid arthritis and osteoarthritis, wherein a pharmaceutically acceptable additive is added to the above-described composition.

The present invention may also provide a functional health food composition for helping with joint health or preventing, alleviating or treating rheumatoid arthritis and osteoarthritis, wherein a sitologically acceptable additive is added to the above-described composition.

### Advantageous Effects

The composition according to the present invention is not toxic to macrophages and rat chondrocytes.

The composition according to the present invention has the effect of inhibiting the mRNA and protein expressions of TNF-α, IL-1β and IL-6, which are proinflammatory cytokines.

The composition according to the present invention has the effect of inhibiting the activities of MMP-2 and MMP-9 in rat chondrocytes and an osteoarthritis animal model.

The composition according to the present invention has the effect of enhancing the activities of collagen type-II, SOX 9 and aggrecan in rat chondrocytes.

The composition according to the present invention has the effects of repairing damage to knee joint cartilage and reducing the loss of proteoglycans.

The composition according to the present invention has the effects of normally restoring the structural change in tibial epiphysis caused by MIA (monosodium iodoacetate).

The composition according to the present invention has the effect of relieving inflammation and pain in arthritis patients.

### DESCRIPTION OF DRAWINGS

FIG. 1 shows the results of measuring cytotoxicity in mouse J774A.1 cells incubated with varying concentrations of HPL-4 for 24 hours;

FIG. 2 shows the results of measuring cytotoxicity in mouse J774A.1 cells incubated with varying concentrations of various solvent fractions of HPL-4 for 24 hours;

FIG. 3 shows the results of measuring cytotoxicity in mouse J774A.1 cells incubated with varying concentrations of a chloroform fraction of HPL-4 for 24 hours;

FIG. 4 shows the results of RT-PCR performed to measure the inhibitory effects of varying concentrations of a chloroform fraction of HPL-4 on the mRNA and protein expressions of proinflammatory cytokines;

FIG. 5 shows the results of measuring the inhibitory effects of varying concentrations of a chloroform fraction of HPL-4 on the mRNA expression of TNF-α;

FIG. 6 shows the results of measuring the inhibitory effects of varying concentrations of a chloroform fraction of HPL-4 on the mRNA expression of IL-1β;

FIG. 7 shows the results of measuring the inhibitory effect of a chloroform fraction of HPL-4 on the mRNA expression of IL-6;

FIG. 8 shows the results of measuring the inhibitory effect of a chloroform fraction of HPL-4 on the mRNA expression of COX-2;

FIG. 9 shows the results of measuring the inhibitory effect of a chloroform fraction of HPL-4 on the mRNA expression of iNOS;

FIG. 10 shows the results of measuring the inhibitory effect of a chloroform fraction of HPL-4 on the mRNA expression of TNF;

FIG. 11 shows the results of measuring the inhibitory effect of a chloroform fraction of HPL-4 on the mRNA expression of IL-6;

FIG. 12 shows the results of measuring the cytotoxicity of HPL-4 in rat chondrocytes;

FIG. 13 shows the results of measuring the inhibitory effects of HPL-4 on the activities of MMP-2 and MMP-9 in rat chondrocytes;

FIG. 14 shows the results of measuring the effects of HPL-4 on the enhancement of activities of collagen type-II, SOX 9 and aggrecan in rat chondrocytes;

FIG. 15 shows the results of measuring the inhibitory effects of HPL-4 on the activities of MMP-2 and MMP-9 in the rat knee joint;

FIG. 16 shows the results of measuring the effect of HPL-4 on the protection of knee joint cartilage in the rat knee joints stained by H & E staining method and Safranin O/fast green staining;

FIG. 17 shows the results of evaluating the action of HPL-4 in the rat knee joint by evaluating histopathological items;

FIG. 18 shows the results of measuring the cartilage protective effect of HPL-4 by photographing the knee joint cartilages of osteoarthritis-induced rats by micro-computed tomography;

FIG. 19 shows the results of measuring the cartilage protective effect of HPL-4 by photographing the trabeculae of tibial epiphysis of osteoarthritis-induced rats by micro-computed tomography;

FIG. 20 shows the results of measuring the anti-inflammatory effects of HPL-4 in the right paws of osteoarthritis-induced rats; and

FIG. 21 shows the results of measuring the pain-relieving effects of HPL-4 in the right paws of osteoarthritis-induced rats.

### BEST MODE

The terms and words used in the present specification and the claims should not be interpreted as being limited to typical meanings or dictionary definitions, but should be interpreted as having meanings and concepts consistent with the technical scope of the present invention, based on the principle that an inventor can appropriately define the concept of the terms to describe his or her invention in the best way.

Accordingly, the embodiments and examples described in the specification and the particulars illustrated in the drawings are merely the most preferable exemplary embodiments of the present invention, but do not represent all the technical spirit of the present invention. Therefore, it should be understood that various equivalents and modifications that can replace the above embodiments, examples and particulars may be present at the time at which the present application is filed.

### Embodiment 1

### Composition for Helping with Joint Health or Preventing, Alleviating or Treating Rheumatoid Arthritis and Osteoarthritis

1) \textcircled{1} Kalopanacis Cortex, Chaenomelis Fructus, Angelicae Gigantis, Zingiberis Rhizoma Crudus, Raphani Semen, and Saururi Herba were prepared, and a 10-fold volume of purified water was added to each of the prepared plant materials.

\textcircled{2} Each of the plant materials was extracted once under reflux at a temperature of 95 to 100° C. for 2-4 hours to prepare an extract of each of the plant materials.

\textcircled{3} The prepared extracts are mixed with one another at a mixing ratio of 150-250 parts by weight of the Kalopanacis Cortex extract: 650-750 parts by weight of the Chaenomelis Fructus extract: 650-750 parts by weight of the Angelicae Gigantis extract: 50-150 parts by weight of the Zingiberis Rhizoma Crudus extract: 150-250 parts by weight of the Saururi Herba extract, and the mixture is used as an active ingredient.

Depending on design conditions, a dry extract obtained by concentrating the mixture under reduced pressure and drying the concentrate may also be used as an active ingredient. An organic solvent may be used in place of purified water in the extraction process, and an extract of each of the plant materials may be prepared by performing extraction twice or more, and may also be fractionated after the extraction process. In addition, after extracts of the plant materials are prepared, the extracts may be filtered, and the filtrates may be mixed with one another. The mixture may be subjected to the additional process of concentrating or drying it or concentrating and drying it. Furthermore, extracts of the plant materials are not mixed with one another immediately after the preparation thereof, but the extracts may be concentrated under reduced pressure and dried to prepare dry extracts, and the dry extracts may be mixed with one another to prepare a dry extract mixture. The dry extract mixture may be used as an active ingredient.

2) \textcircled{1} 150-250 parts by weight of Kalopanacis Cortex, 650-750 parts by weight of Chaenomelis Fructus, 650-750 parts by weight of Angelicae Gigantis, 50-150 parts by weight of Zingiberis Rhizoma Crudus and 150-250 parts by weight of Saururi Herba are mixed with one another, and a 10-fold volume of purified water is added to the mixture.

\textcircled{2} The mixture is extracted once under reflux at a temperature of 95 to 100° C. for 2-4 hours to prepare a mixed extract, and the mixed extract is used as an active ingredient.

It will be apparent that the extraction solvent, the number of extractions, the extraction time, and the addition of an additional process after the extraction process may be changed depending on design conditions.

3) A pharmaceutically acceptable carrier, excipient or diluent is added to the active ingredient, prepared in the above method 1) or 2), thereby providing a medicine for preventing or treating skin aging formulated in a pharmaceutical unit dosage form and thus achieving the above-described objects.

In this case, examples of the carrier, excipient or diluent include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxylbenzoate, talc, magnesium stearate, and mineral oil.

Furthermore, the pharmaceutical dosage form may be used in the form of a pharmaceutically acceptable salt, and may also be used alone or in combination with other pharmaceutically active compounds.

Furthermore, the active ingredient prepared in the above method 1) or 2) may be formulated with commonly used diluents or excipients, such as fillers, extenders, binders, wetting agents, disintegrants, surfactants, etc.

Moreover, the pharmaceutical dosage form may be prepared in oral dosage forms, including powders, granules, tablets, capsules, suspensions, emulsions, syrup and aerosol, as well as suppositories and sterile injectable solutions.

Solid formulations for oral administration may be prepared by adding at least one excipient, for example, starch, calcium carbonate, sucrose, lactose, gelatin or the like, to the extract. In addition to simple excipients, lubricants such as magnesium stearate or talc may also be used.

Formulations for parenteral administration include sterilized aqueous solutions, non-aqueous solutions, suspensions, emulsions, freeze-dried preparations, and suppositories.

As non-aqueous solvents or suspending agents, propylene glycol, polyethylene glycol, plant oils such as olive oil, injectable esters such as ethyl oleate, and the like can be used.

As the base of the suppositories, witepsol, Macrogol, Tween 61, cacao butter, laurin fat, glycerogelatin and the like can be used.

The preferred dose of the active ingredient according to the present invention varies depending on the patient's condition and bodyweight, the severity of the disease, the patient's age and gender, the form of drug, and the route and period of administration, but can be appropriately selected by those skilled in the art.

The composition of the present invention may be administered to mammals, including rats, mice, livestock and humans, by various routes. All routes of administration can be contemplated and include. For example, the composition for alleviating or treating rheumatoid arthritis and osteoarthritis according to the present embodiment may be administered via oral, tissue, rectal, intravenous, intramuscular or subcutaneous routes.

Meanwhile, a food additive may be added to the active ingredient prepared according to the above method 1) or 2), thereby preparing a functional health food composition for preventing, alleviating or treating rheumatoid arthritis and osteoarthritis and thus achieving the above-described objects. Examples of foods, to which the extract may be added, include various foods, gums, teas, vitamin complexes, and functional health foods. In addition, the extract may be added to foods or beverages for preventing or alleviating skin aging.

In this case, the content of the extract in the food or the beverage may be 0.01-20 wt % based on the total weight of the food. For the health beverage composition, the extract may be added in an amount of 0.02-5 g, and preferably 0.3-1 g, based on 100 ml of the health beverage composition.

In addition to the extract, other components may be contained in the functional health food composition, and these components are not specifically limited. Specifically, the functional health food composition of the present invention may contain additional components, including various flavoring agents or natural carbohydrates, which are contained in conventional beverages.

Examples of the natural carbohydrates include sugars such as monosaccharides, for example, glucose, fructose, etc., disaccharides, for example, maltose, sucrose, etc., or polysaccharides, for example, dextrin, cyclodextrin, etc., and sugar alcohols such as xylitol, sorbitol, erythritol, etc. In addition to these carbohydrates, a natural flavoring agent (thaumatin, stevia extract (e.g., rebaudioside A, glycyrrhizin, etc.)) or a synthetic flavoring agent (saccharin, aspartame, etc.) may advantageously be used.

The natural carbohydrate is generally used in an amount of about 1-20 g, and preferably about 5-12 g, based on 100 ml of the composition of the present invention.

In addition, the composition of the present invention may further contain one or more selected from among various nutrients, vitamins, minerals (electrolytes), synthetic and natural flavoring agents, colorants, fillers (cheese, chocolate, etc.), pectic acid and its salts, alginic acid and its salts, organic acids, protective colloidal thickeners, pH adjusting agents, stabilizers, preservatives, glycerin, alcohol, and carbonating agents for carbonated beverages.

Additionally, the composition of the present invention may contain fruit flesh that is used for the preparation of natural fruit juice, fruit juice beverages or vegetable beverages.

These additives may be used alone or in combination. The content of these additives in the composition of the present invention is not so critical, but is generally in the range of 0 to 20 parts by weight based on 100 parts by weight of the composition.

### Preparation Example 1

### Composition for Helping with Joint Health or Preventing, Alleviating or Treating Rheumatoid Arthritis and Osteoarthritis

\textcircled{1} Kalopanacis Cortex, Chaenomelis Fructus, Angelicae Gigantis, Zingiberis Rhizoma Crudus, Raphani Semen and Saururi Herba were prepared, and a 10-fold volume of purified water was added to each of the prepared plant materials.

\textcircled{2} Each of the prepared plant materials was extracted twice under reflux at a temperature of 95 to 100° C. for 2-4 hours to prepare extracts.

\textcircled{3} Each of the extracts was filtered through a 25-μm filter to prepare filtrates.

\textcircled{4} Each of the filtrates was concentrated under reduced pressure at a temperature of 60° C. or lower to prepare dry extracts.

\textcircled{5} The dry extracts were mixed with one another at a weight ratio of 2 (dry extract of Kalopanacis Cortex):7 (dry extract of Chaenomelis Fructus):7 (dry extract of Zingiberis Rhizoma Crudus):1 (dry extract of Raphani Semen):2 (dry extract of Saururi Herba) to prepare a mixed dry extract for use as an active ingredient.

### Test Example 1

### Cytotoxicity in Macrophages

It is to be noted that Test Examples 1 to 9 were conducted using the dry mixed extract prepared in Preparation Example 1 above as a main test sample.

In addition, it is to be noted that “HPL-4” or “HPL-04”, described in Test Examples 1 to 9 and shown in the figures, is the name temporarily designated by the applicant during the tests, and refers to the mixture prepared in Preparation Example 1.

1-1: Test Process

In order to examine the effect of HPL-4 on the proliferation of mouse macrophage J774A.1 cells, a CCK-8 assay was performed.

First, cells were incubated with varying concentrations (1, 10, 100, 200 and 500 μg/ml) of HPL-4 for 24 hours.

Then, HPL-4 was fractionated with four fractions (chloroform (CHCl₃), ethyl acetate, butanol and water fractions), and cells were incubated with varying concentrations (10 and 100 μg/ml) of each of the fractions for 24 hours.

In addition, cells were incubated with varying concentrations (10, 25, 50 and 100 μg/ml) of a chloroform fraction of HPL-4 for 24 hours.

1-2: Test Results

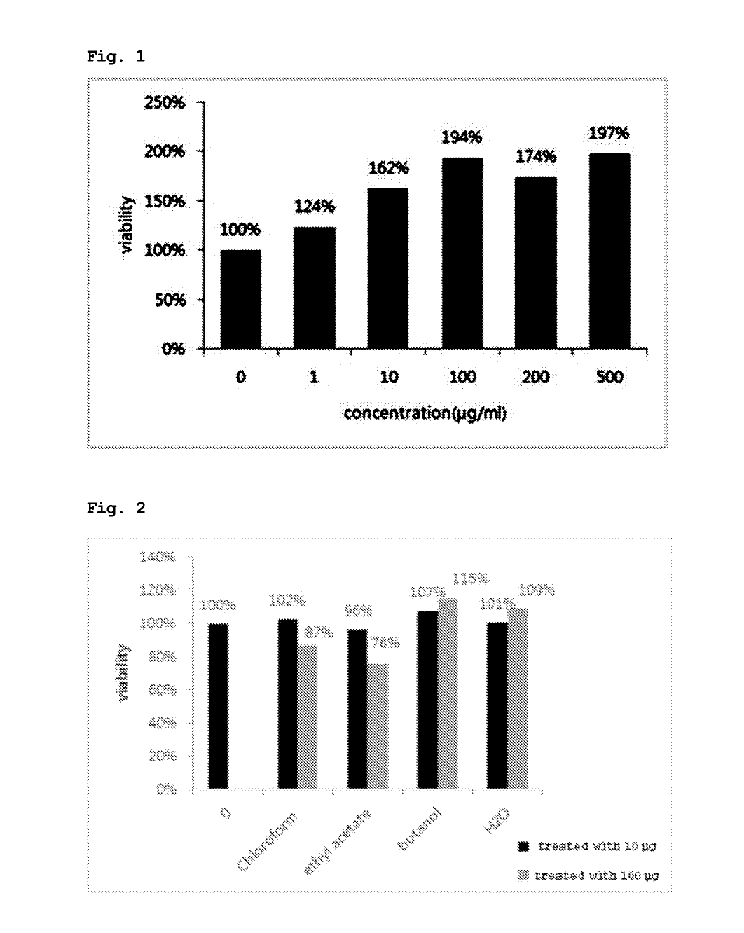

FIG. 1 shows the results of measuring cytotoxicity in mouse J774A.1 cells incubated with varying concentrations of HPL-4 for 24 hours.

As can be seen therein, when the cells were incubated with varying concentrations of HPL-4 for 24 hours, HPL-4 did not show cytotoxicity and the inhibition of cell proliferation at all the concentrations, unlike the control. In particular, it was shown that HPL-4 stimulated the proliferation of the J774A.1 cells in a concentration-dependent manner.

FIG. 2 shows the results of measuring cytotoxicity in mouse J774A.1 cells incubated with varying concentrations of various solvent fractions of HPL-4 for 24 hours.

As can be seen therein, when the cells were incubated with varying concentrations of various solvent fractions of HPL-4 for 24 hours, all the solvent fractions did not inhibit cell proliferation at a concentration of 100 μg/ml, unlike the control. However, at a concentration of 100 μg/ml, the chloroform fraction and the ethyl acetate fraction reduced cell proliferation by 13% and 24%, respectively, even though no cell death appeared. Thus, the maximum concentration of HPL-4 was set at 100 μg/ml at which no toxicity appeared, and then a subsequent test was conducted in order to examine the anti-inflammatory effect of HPL-4.

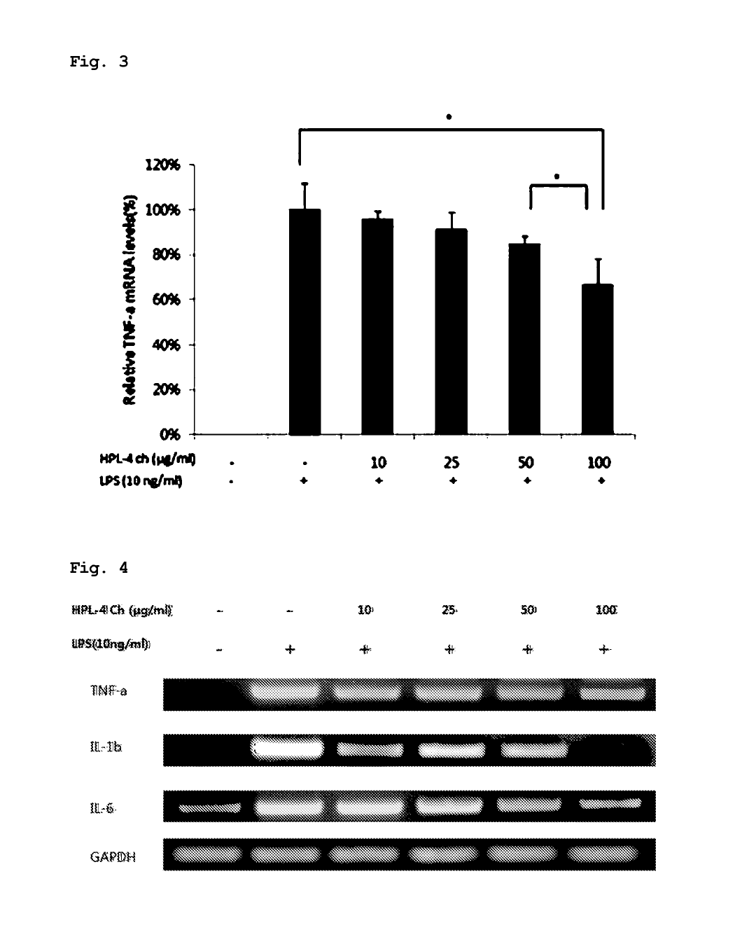

FIG. 3 shows the results of measuring cytotoxicity in mouse J774A.1 cells incubated with varying concentrations of a chloroform fraction of HPL-4 for 24 hours.

As can be seen therein, when cells were incubated with varying concentrations of a chloroform fraction of HPL-4 for 24 hours, the proliferation of the cells was not inhibited even at a HPL-4 concentration of 50 μg/ml, unlike the control. However, at a concentration of 100 μg/ml, the proliferation of the cells was reduced by 13%, even though no cell death appeared. Thus, the maximum concentration of HPL-4 was set at 100 μg/ml at which no toxicity appeared, and then a subsequent test was conducted in order to examine the anti-inflammatory effect of HPL-4.

### Test Example 2

### Inhibitory Effects of HPL-4 on Expression of Proinflammatory Cytokines

2-1: Test Process

The mRNA and protein expressions of TNF-α, IL-1β and IL-6, which are proinflammatory cytokines formed by LPS stimulation, were measured using real-time (RT) PCR and an ELISA kit.

2-2: Test Results

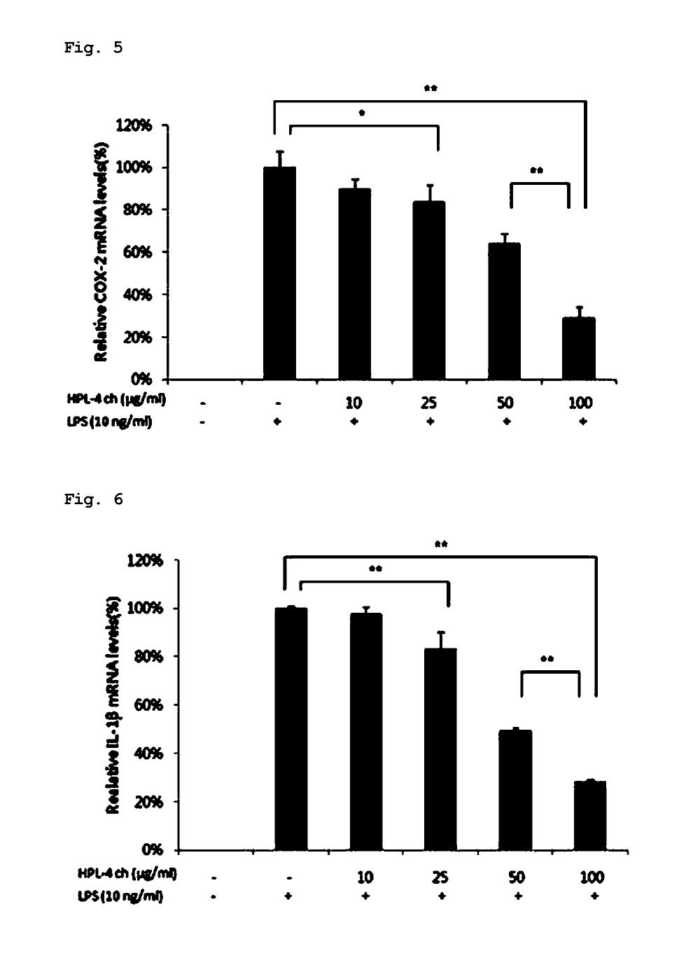

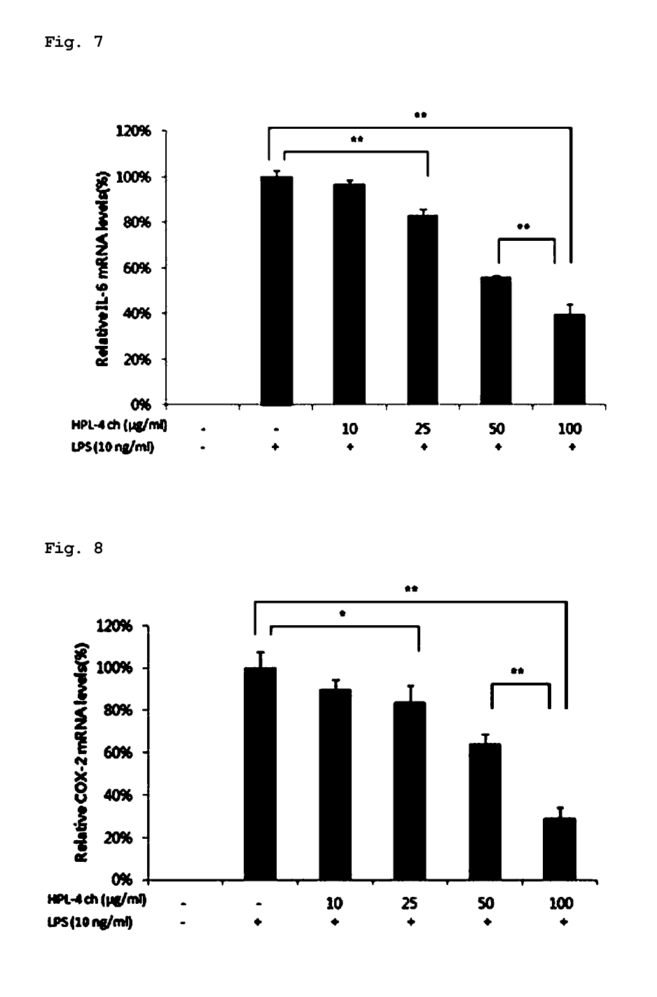

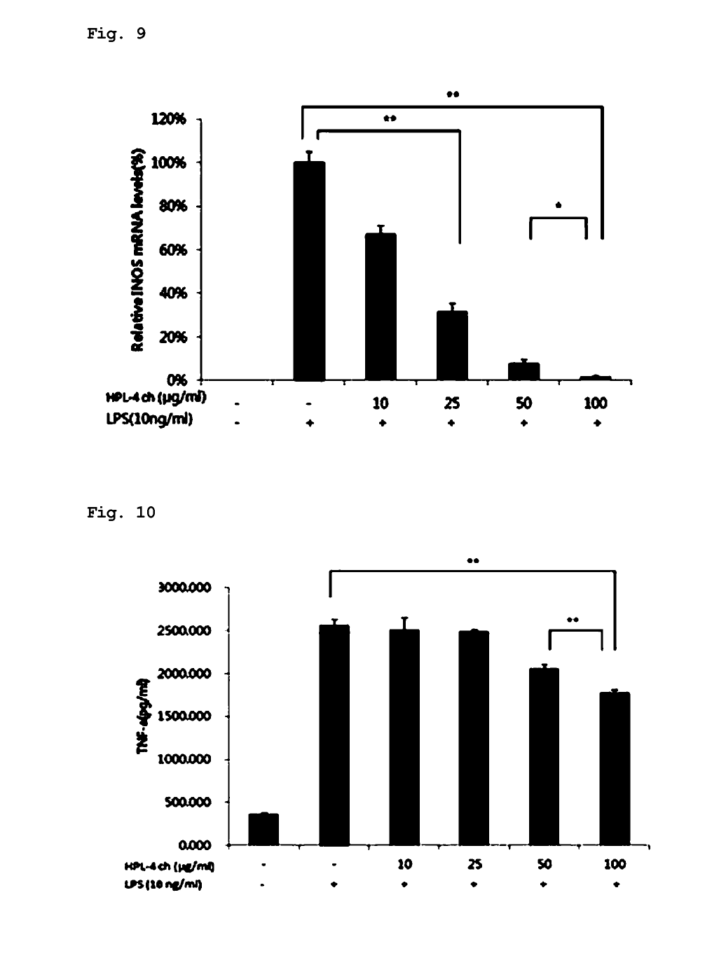

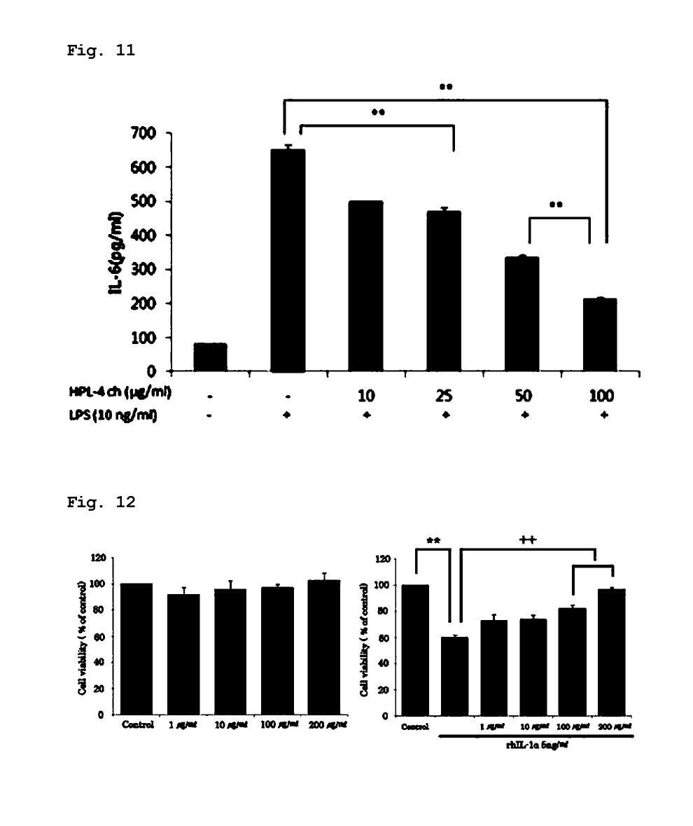

FIG. 4 shows the results of RT-PCR performed to measure the inhibitory effects of varying concentrations of a chloroform fraction of HPL-4 on the mRNA and protein expressions of proinflammatory cytokines; FIG. 5 shows the results of measuring the inhibitory effects of varying concentrations of a chloroform fraction of HPL-4 on the mRNA expression of TNF-α; FIG. 6 shows the results of measuring the inhibitory effects of varying concentrations of a chloroform fraction of HPL-4 on the mRNA expression of IL-1β; FIG. 7 shows the results of measuring the inhibitory effect of a chloroform fraction of HPL-4 on the mRNA expression of IL-6; FIG. 8 shows the results of measuring the inhibitory effect of a chloroform fraction of HPL-4 on the mRNA expression of COX-2; FIG. 9 shows the results of measuring the inhibitory effect of a chloroform fraction of HPL-4 on the mRNA expression of iNOS; FIG. 10 shows the results of measuring the inhibitory effect of a chloroform fraction of HPL-4 on the mRNA expression of TNF; and FIG. 11 shows the results of measuring the inhibitory effect of a chloroform fraction of HPL-4 on the mRNA expression of IL-6. As can be seen therein, when the mRNA and protein expressions of proinflammatory cytokines were measured and the expressions of TNF-α, IL-1β and IL-6 in J774A.1 cells treated with LPS were analyzed by RT-PCR, the mRNA expressions of the proinflammatory cytokines were significantly increased by LPS, and were reduced by treatment with HPL-4 in a manner dependent on the concentration of HPL-4.

In particular, in the RT-PCR analysis, it was shown that the mRNA expression levels of TNF-α, IL-1β and IL-6 were reduced by 32%, 44% and 51%, respectively, at a HPL-4 concentration of 100 μg/ml.

COX-2 is an enzyme that is involved in the production of the inflammation-associated factor PGE2. The effect of a chloroform fraction of HPL-4 on the LPS-induced expression of COX-2 in J774A.1 cells was examined. As a result, it was shown that the expression of COX-2 in an LPS-treated group significantly increased compared to that in a group not treated with LPS and that the mRNA expression of COX-2 in a group treated with LPS+HPL-4 was significantly inhibited at a HPL-4 concentration of 50 μg/ml or higher. In particular, it was shown that, at a HPL-4 concentration of 100 μg/ml, the mRNA expression of COX-2 was reduced by up to 71%.

iNOS (inducible nitric oxide synthase) is an enzyme that forms NO, and an increase in the expression thereof is involved in inflammatory reactions and tissue damage. The production of NO in macrophages is associated with the expression of iNOS, and is induced by stimulation with cell wall substances (such as LPS) of gram negative bacteria, or cytokines. Thus, in this experiment, J774A.1 cells treated with LPS were incubated with varying concentrations of a chloroform fraction of HPL-4, and then the inhibitory effect of the chloroform fraction of HPL-4 on the expression of iNOS in the cells was analyzed. As a result, it was shown that the expression of iNOS in the group treated with LPS significantly increased compared to that in the control group not treated with LPS, and the expression of iNOS in the group treated with LPS plus the chloroform fraction of HPL-4 decreased in a manner dependent on the concentration of HPL-4. In particular, it was shown that, at a HPL-4 concentration of 100 μg/ml, the expression of iNOS decreased to a level similar to that in the control group, indicating that HPL-4 effectively inhibits the expression of iNOS.

Protein secretion levels of TNF-α, IL-1 and IL-6 in a cell culture medium treated with HPL-4 were measured using an ELISA kit. As a result, it was shown that the secretion of TNF-α and IL-6 in the group treated with the HPL-4 extract was significantly inhibited in a manner dependent on the concentration of HPL-4, compared to that in the group treated with LPS alone. However, the secretion level of IL-1 protein was not detected in the LPS-treated cells, and thus was excluded from the analysis.

### Test Example 3

### Measurement of Cell Viability in Animal Chondrocyte Model

3-1: Test Process

MC3T-3E1 osteoblasts and articular chondrocytes isolated from the joints of SD rats were incubated.

Normal chondrocytes were treated with varying concentrations of HPL-4, and cytotoxicity in the cells was measured. At 1 hour after pretreatment with HPL-4, the cells were treated with rhIL-1α (5 ng/ml), and cytotoxicity in the cells was measured. In this case, the cells were treated with HPL-4 at concentrations of 1, 10, 100 and 200 μg/ml.

All the test groups were analyzed by an MTT assay to measure cytotoxicity in the chondrocyte animal model.

3-2: Test Results

FIG. 12 shows the results of measuring the cytotoxicity of HPL-4 in rat chondrocytes.

The results of evaluation of cytotoxicity of HPL-4 indicated that HPL-4 was not toxic at a concentration of 1-200 μg/ml.

In addition, it was shown that the viability of cells in the group treated with 5 ng/ml of rh IL-1 alone significantly decreased and that the viability of cells in the group treated with 1-200 μg/ml of HPL-4 increased in a manner dependent on the concentration of HPL-4, and the viability of cells in the group treated with 200 μg/ml of HPL-4 significantly increased. Thus, it can be concluded that HPL-4 inhibits cell death induced by rh IL-1.

### Test Example 4

### Effect of HPL-4 on Inhibition of Cartilage Absorption in Chondrocytes

4-1: Test Process

Cells were prepared in the same manner as in Example 3 and were pretreated with 10, 100 and 200 μg/ml of HPL-4.

4-2: Test Results

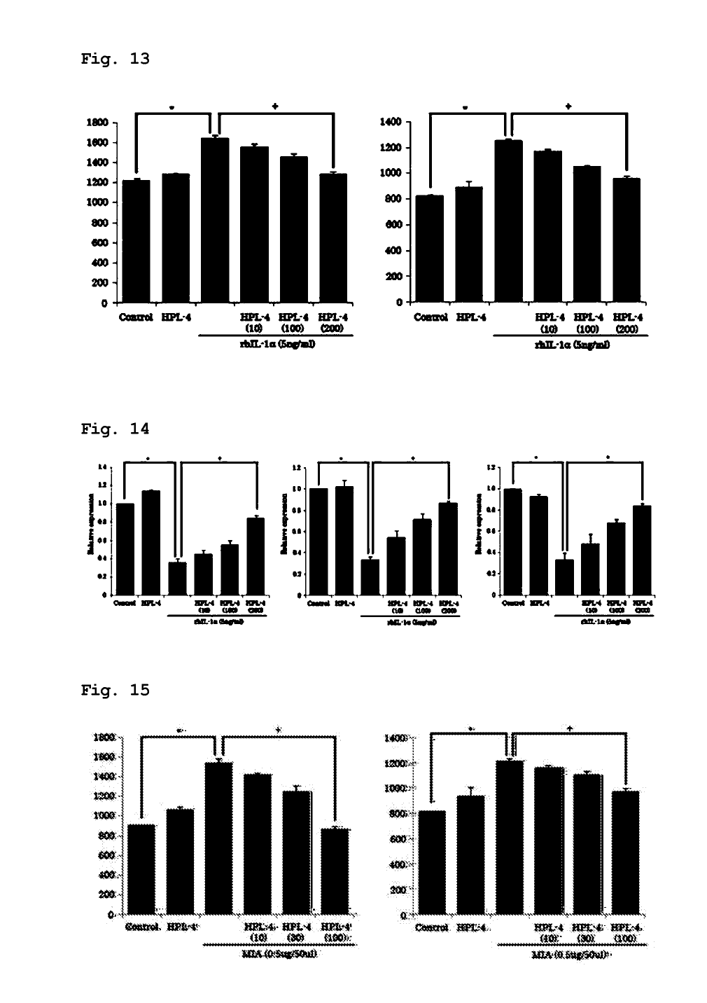

FIG. 13 shows the results of measuring the inhibitory effects of HPL-4 on the activities of MMP-2 and MMP-9 in rat chondrocytes.

As a result, it was shown that treatment with HPL-4 alone had no effect on the activities of MMP-2 and MMP-9 and that, in the group having increased activities of MMP-2 and MMP-9 as a result of treatment with rhIL-1α (5 ng/ml) alone, the activities of MMP-2 and MMP-9 was decreased by 1-hour pretreatment with HPL-4 in a manner dependent on the concentration of HPL-4. This indicates that HPL-4 inhibits the rhIL-1α-induced activities of MMP-2 and MMP-9 that are cartilage absorption factors.

### Test Example 5

### Effect of HPL-4 on Stimulation of Cartilage Formation in Animal Chondrocyte Model

5-1: Test Process

SD rats were divided into a total of 6 groups: a control group administered with collagen type-II, SOX 9 and aggrecan and treated with anything; a group treated with HPL-4 along; a group treated with rhIL-1α; and groups treated with rhIL-1α and HPL-4. In this case, collagen type-II, SOX 9 and aggrecan are chondrogenic factors. In the groups treated rhIL-1α and HPL-4 (pretreated with HPL-4 for 1 hour), the concentration of HPL-4 was set at 10, 100 and 200 μg/ml.

5-2: Test Results

FIG. 14 shows the results of measuring the effects of HPL-4 on the enhancement of activities of collagen type-II, SOX 9 and aggrecan in rat chondrocytes.

The activities of collagen type-II, SOX 9 and aggrecan that are chondrogenic factors were measured. As a result, it was shown that in the group treated with 5 ng/ml of rh IL-1 alone, the activities of collagen type-II 2, SOX 9 and aggrecan that are chondrogenic factors significantly decreased, and in the group pretreated with 10-200 μg/ml of HPL-4 for 1 hour, the chondrogenic factors were significantly restored in a manner dependent on the concentration of HPL-4.

Thus, it can be concluded that HPL-4 increases the activities of the chondrogenic factors, collagen type-II, SOX 9 and aggrecan, which are inhibited by rh IL-1.

### Test Example 6

### Effect of HPL-4 on Inhibition of Cartilage Absorption in Animal Model

6-1: Test Process

MIA (monosodium iodoacetate, I2512) (0.5 μg/50 μl) was injected into the joints of SD rats through the patella ligament of the knee joints to construct an osteoarthritis model. HPL-4 was administered at doses of 10, 30 and 100 mg/kg for 4 weeks.

6-2: Test Results

FIG. 15 shows the results of measuring the inhibitory effects of HPL-4 on the activities of MMP-2 and MMP-9 in the rat knee joint.

As a result, it was observed that the activities of MMP-2 and MMP-9 that are cartilage absorption factors were significantly increased by administration of MIA, but the activities of MMP-2 and MMP-9 in the group administered with 10-100 mg/kg of HPL-4 for 4 weeks decreased in a manner dependent on the concentration of HPL-4.

In particular, it was observed that the activities of MMP-2 and MMP-9 in the group treated with 100 mg/kg of HPL-4 significantly decreased, and the activities of MMP-2 and MMP-9 in the group administered with 100 mg/kg of HPL-4 did not change.

Thus, it can be concluded that HPL-4 inhibits the activities of the cartilage absorption factors (MMP-2 and MMP-9) in MIA-induced osteoarthritis.

### Test Example 7

### Effect of HPL-4 on Inhibition of Knee Joint Damage in Animal Model

7-1: Test Process

As arthritis processes, the spacing between joints become progressively narrower, and sclerosis of articular cartilage occurs. In addition, when the progression of arthritis continues, bone spurs are formed at the edge of the articular surface, and the articular surface becomes irregular. Such aspects of progression of arthritis were examined.

(1) Knee joint sections were obtained from the osteoarthritis model induced by MIA (monosodium iodoacetate, I2512) (0.5 μg/50 μl) and were observed by staining.

In this case, the observation of knee joint sections by staining was performed in the following manner.

The knee joint tissue of SD rats was fixed in 10% neutral formalin, and then embedded in paraffin and cut into 5 μm sections. The sections were divided into 4 groups: a control group; a group administered with HPL-4 alone; a group administered with MIA and vehicle; and a group administered with MIA and HPL-4. The groups were allowed to stand for 4 weeks after administration of the drugs. HPL-4 was administered at a dose of 100 mg/kg.

All the test groups were stained by H & E (hematoxilin & eosin) staining and Safranin O-fast green staining, and then observed with an optical microscope. In this case, Safranin O-fast green staining is frequently used to discriminate among cartilage, mucin and mast cells in tissue. The cell nucleus is stained with red or black, cartilage is stained with orange or red, and the cytoplasm is stained with green or blue. Positive Safranin O red staining of bone sections in the growth period in which bone tissue is actively produced means PGs (sulfated proteoglycans) and can also be observed even when the regeneration of bone tissue in osteoarthritis is active.

(2) In addition, the group administered with HPL-4 for 4 weeks was evaluated by histopathological items.

(3) Furthermore, the knee joint cartilage and tibial epiphysis in the group administered with HPL-4 for 4 weeks were observed by micro CT.

7-2: Test Results

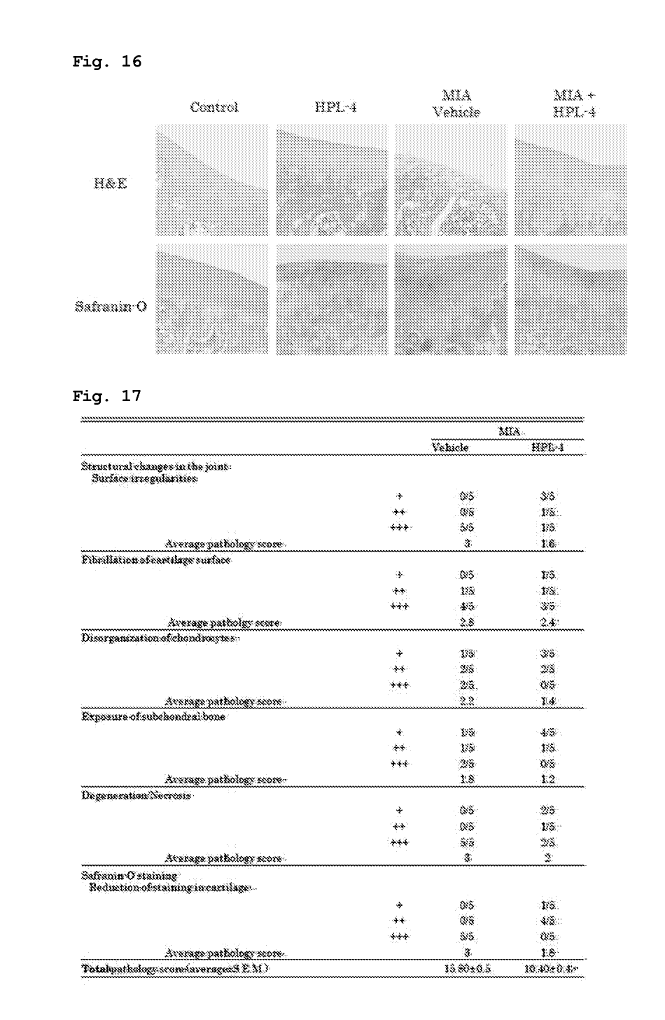

(1) FIG. 16 shows the results of measuring the effect of HPL-4 on the protection of knee joint cartilage in the rat knee joints stained by H & E staining method and Safranin 0/fast green staining.

As a result, the group treated with HPL-4 for 4 weeks did not show any difference from the control group. It could be seen that in the knee joint cartilage of the group treated with MIA alone, bone erosion and cartilage destruction were observed, but in the group administered with HPL-4 for 4 weeks after injection of MIA, cartilage destruction was inhibited.

In addition, cartilage destruction and the content of proteoglycans were analyzed by Safranin O-fast green staining. The results of observation of cartilage erosion indicated that all the layers of joint cartilage in the control group were stained red, indicating a high content of proteoglycans, but in the group administered with MIA, joint cartilage defects or erosion was severe, and the loss of proteoglycans increased compared to that in the control group. In addition, it was observed that in the group administered with HPL-4, damage to cartilage was repaired compared to that in the control group, and the loss of proteoglycans decreased compared to that in the control group. Thus, it can be concluded that HPL-4 protects cartilage destructed by MIA.

(2) FIG. 17 shows the results of evaluating the action of HPL-4 in the rat knee joint by evaluating histopathological items.

As can be seen therein, the total pathologic finding score was 15.80±0.5 for the group administered with MIA and vehicle and 10.40±0.4 for the group administered with HPL-4 alone.

Thus, it can be concluded that HPL-4 significantly protects cartilage destructed by MIA.

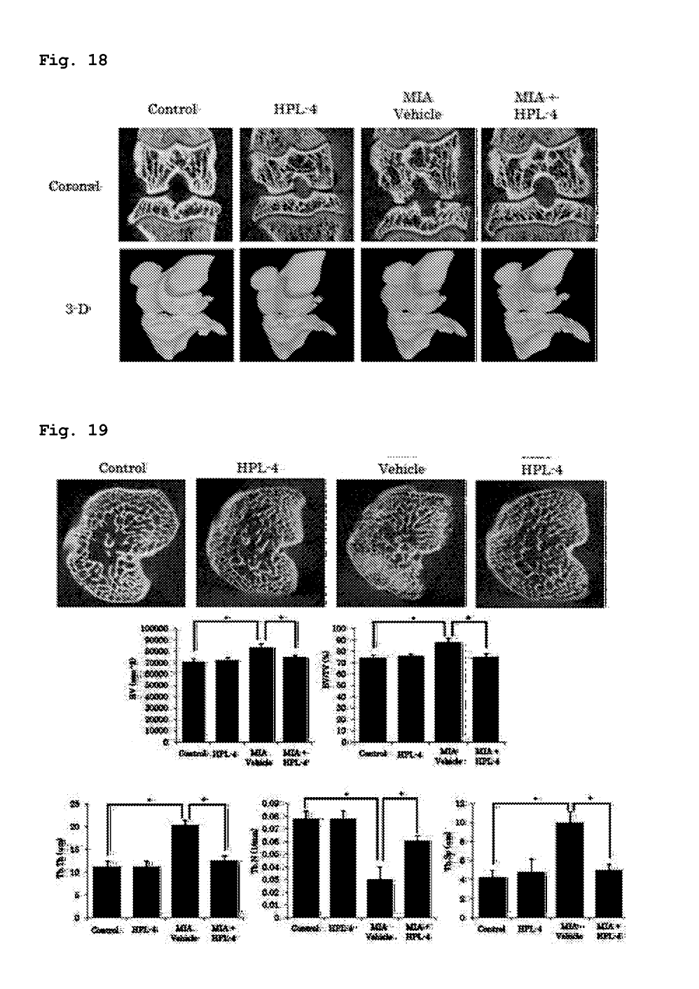

(3) FIG. 18 shows the results of measuring the cartilage protective effect of HPL-4 by photographing the knee joint cartilages of osteoarthritis-induced rats by micro-computed tomography; and FIG. 19 shows the results of measuring the cartilage protective effect of HPL-4 by photographing the trabeculae of tibial epiphysis of osteoarthritis-induced rats by micro-computed tomography.

In FIG. 19, BV represents bone volume; BV/TV represents bone volume fraction; Tb.Th represents trabecular thickness; Tb.N represents trabecular number; and Tb.Sp represents trabecular spacing.

As can be seen in FIGS. 18 and 19, the CT images of the normal group and the group administered with HPL-4 alone showed no abnormal finding. However, in the group administered with MIA alone, it was observed that severe cartilage damage and destruction occurred, and the outer surface of cartilage was severely destructed. However, in the group administered with HPL-4 for 4 weeks, it could be seen that cartilage damage decreased and the rough surface in the cartilage cross-section decreased compared to that in the MIA-administered group.

In addition, trabecular index values obtained from the cylindrical VOI of tibial epiphysis in cross-sectional images of micro-CT were compared between the knee joint of the group administered with MIA alone and the knee joint of the group administered with HPL-4. As a result, it could be seen that, in the group administered with MIA alone, BV, BV/TV and Tb.Th values increased, and Tb.N and Tb.Sp values decreased. However, in the group administered with HPL-4 after administration of MIA, BV, BV/TV and Tb.Th values decreased to values close to those in the control group, and Tb.N and Tb.Sp values increased to values close to those in the control group.

Thus, it can be concluded that HPL-4 inhibits the changes in various index factors of tibial epiphysis induced by MIA.

### Test Example 8

### Anti-Inflammatory Effect of HPL-4 in Animal Model

8-1: Test Process

SD rats were divided into the following groups: a control group; a group administered with celecoxib; and a group administered with HPL-4. The control group was administered with vehicle, the celecoxib-administered group was administered with celecoxib, and the HPL-4-administered group was administered with varying doses of HPL-4. In this case, the HPL-4-administered group was administered with 10, 30 and 100 mg/kg of HPL-4.

At 1 hour after administration of the drug to the test group, 200 μl (20 mg/ml) of zymosan was administered intracutaneously to the rats. From 1 hour after the administration of zymosan, paw edema was observed over 5 hours.

8-2: Test Results

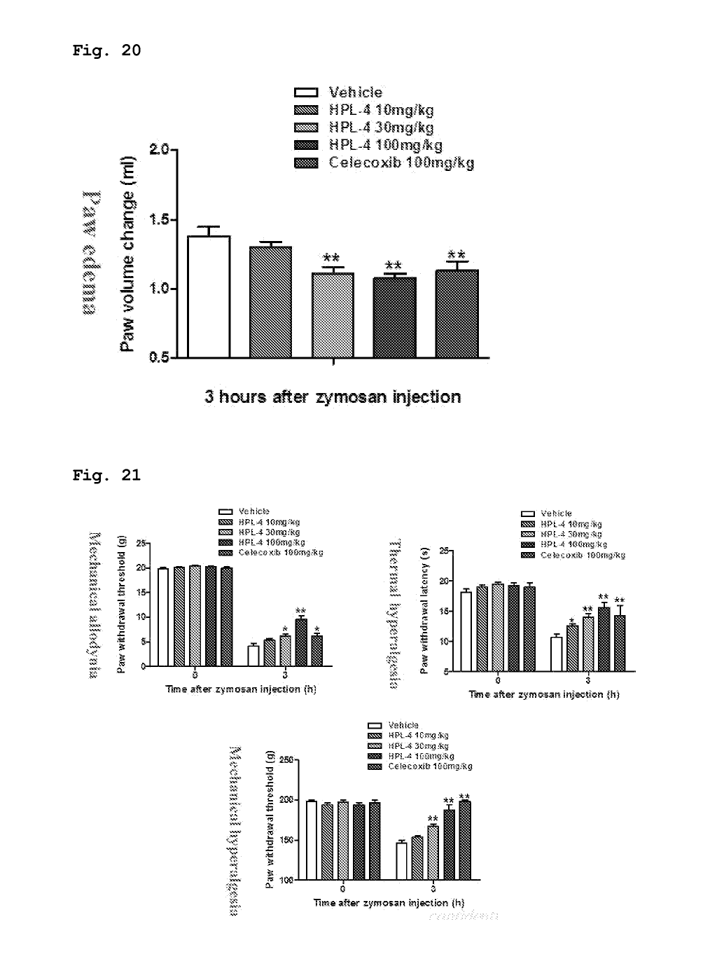

FIG. 20 shows the results of measuring the anti-inflammatory effects of HPL-4 in the right paws of osteoarthritis-induced rats.

As can be seen in FIG. 20, the volume of paw edema in the control vehicle group was close to about 1.5 ml. It could be seen that, in the HPL-4-administered group, the volume of paw edema gradually decreased as the dose of HPL-4 increased from 10 mg/kg to 30 and 100 mg/kg. In particular, the volume of paw edema in the groups administered with 30 or 100 mg/kg of HPL-4 was smaller than that in the control group administered with 100 mg/kg of celecoxib. Thus, it can be concluded that HPL-4 has an anti-inflammatory effect.

### Test Example 9

### Pain-Relieving Effect of HPL-4 in Animal Model

9-1: Test Process

Preparation for a test was conducted in the same manner as described in Test Example 8.

When inflammatory pain occurs, hypersensitive pain different from normal pain appears. Hypersensitive pain can be classified into hyperalgesia that is stronger than normal pain, and allodynia that is caused by non-noxious stimuli.

In addition, spontaneous pain is often involved, and the site feeling pain may also be expanded to a site around the damage site. Such symptoms also appear upon the onset of arthritis.

Methods employing chemical substances are used to cause hypersensitive pain. When CFA (complete Freund adjuvant), carrageenan, capsaicin, formalin or the like is injected into the paw of rats, characteristic inflammatory reactions such as erythema, edema or hyperalgesia are induced so that hyperalgesia behaviors will appear, and the excitability of dorsal horn neurons that receive pressure from a site having induced inflammation is increased.

After completion of test preparation, a thermal stimulus was applied to the test group at 1 hour after administration of zymosan to induce thermal hyperalgesia. After 3 hours, the paw withdrawal latency was measured.

In addition, a mechanical stimulus was applied to induce mechanical allodynia and mechanical hyperalgesia. After 3 hours, the paw withdrawal threshold was measured.

9-3: Test Results

FIG. 21 shows the results of measuring the pain-relieving effects of HPL-4 in the right paws of osteoarthritis-induced rats.

As can be seen therein, when the thermal stimulus was applied to induce thermal hyperalgesia, the paw withdrawal latency in all the test group was reduced over time.

The paw withdrawal latency was the lowest in the vehicle-administered group, and was higher in the HPL-4-administered group and the celecoxib-administered group than in the vehicle-administered group, suggesting that HPL-4 has a pain-relieving effect.

In particular, in the case of the HPL-4-administered group, the paw withdrawal latency gradually increased with an increase in the dose of HPL-4, and when the dose of HPL-4 was increased to 100 mg/kg, the paw withdrawal latency was higher than that of the celecoxib-administered group.

When the mechanical stimulus was applied to induce mechanical allodynia, the paw withdrawal thresholds in all the test groups were reduced over time.

The paw withdrawal value was the lowest in the vehicle-administered group, and was higher in the HPL-4-administered group and the celecoxib-administered group, suggesting that HPL-4 has a pain-relieving effect.

In particular, in the case of the HPL-4-administered group, the paw withdrawal threshold gradually increased with an increase in the dose of HPL-4, and when the dose of HPL-4 was increased to 100 mg/kg, the paw withdrawal threshold was higher than that of the celecoxib-administered group.

When the mechanical stimulus was applied to induce mechanical hyperalgesia, the paw withdrawal thresholds in the vehicle-administered group and the HPL-4-administered group were reduced over time.

The paw withdrawal threshold was the lowest in the vehicle-administered group, and did not change in the celecoxib-administered group even with the passage of time. In the case of the HPL-4-administered group, the paw withdrawal threshold gradually increased with an increase in the dose of HPL-4, and when the dose of HPL-4 was increased to 100 mg/kg, the paw withdrawal threshold was closest to that of the celecoxib-administered group. Therefore, it can be concluded that HPL-4 has pain-relieving effects against mechanical allodynia, thermal hyperalgesia and mechanical hyperalgesia.

1. A composition for helping with joint health or preventing, alleviating or treating rheumatoid arthritis and osteoarthritis, which contains, as an active ingredient:
an extract mixture obtained by extracting each of Kalopanacis Cortex, Chaenomelis Fructus, Angelicae Gigantis, Zingiberis Rhizoma Crudus, Raphani Semen and Saururi Herba to prepare extracts, and mixing the extracts with one another at a mixing ratio of 150-250 parts by weight of the Kalopanacis Cortex extract: 650-750 parts by weight of the Chaenomelis Fructus extract: 650-750 parts by weight of the Angelicae Gigantis extract: 50-150 parts by weight of the Zingiberis Rhizoma Crudus extract: 50-150 parts by weight of the Raphani Semen extract: 150-250 parts by weight of the Saururi Herba extract; or
a dry extract mixture obtained by drying each of the extracts and mixing the dried extracts with one another at the above mixing ratio; or a mixed extract obtained by mixing Kalopanacis Cortex, Chaenomelis Fructus, Angelicae Gigantis, Zingiberis Rhizoma Crudus, Raphani Semen and Saururi Herba with one another at the above mixing ratio to prepare a mixture, and extracting the mixture.

2. The composition of claim 1, which has effects on inhibition of inflammatory cytokines and matrix metalloproteinases (MMPs), relief of inflammation and pain, enhancement of activity of cartilage formation factors, and normalization of a joint structure.

3. The composition of claim 1, wherein a number of times the extraction is performed when each of the extracts or mixed extract is prepared is two or more.

4-5. (canceled)

6. A pharmaceutical composition for alleviating or treating rheumatoid arthritis and osteoarthritis, which contains, as a pharmaceutically acceptable additive:
an extract mixture obtained by extracting each of Kalopanacis Cortex, Chaenomelis Fructus, Angelicae Gigantis, Zingiberis Rhizoma Crudus, Raphani Semen and Saururi Herba to prepare extracts, and mixing the extracts with one another at a mixing ratio of 150-250 parts by weight of the Kalopanacis Cortex extract: 650-750 parts by weight of the Chaenomelis Fructus extract: 650-750 parts by weight of the Angelicae Gigantis extract: 50-150 parts by weight of the Zingiberis Rhizoma Crudus extract: 50-150 parts by weight of the Raphani Semen extract: 150-250 parts by weight of the Saururi Herba extract; or
a dry extract mixture obtained by drying each of the extracts and mixing the dried extracts with one another at the above mixing ratio; or a mixed extract obtained by mixing Kalopanacis Cortex, Chaenomelis Fructus, Angelicae Gigantis, Zingiberis Rhizoma Crudus, Raphani Semen and Saururi Herba with one another at the above mixing ratio to prepare a mixture, and extracting the mixture.

7. The composition of claim 6, which has effects on inhibition of inflammatory cytokines and matrix metalloproteinases (MMPs), relief of inflammation and pain, enhancement of activity of cartilage formation factors, and normalization of a joint structure.

8. The composition of claim 6, wherein a number of times the extraction is performed when each of the extracts or mixed extract is prepared is two or more.

9. A functional health food composition for helping with joint health or preventing, alleviating or treating rheumatoid arthritis and osteoarthritis, which contains, as a sitologically acceptable additive:
an extract mixture obtained by extracting each of Kalopanacis Cortex, Chaenomelis Fructus, Angelicae Gigantis, Zingiberis Rhizoma Crudus, Raphani Semen and Saururi Herba to prepare extracts, and mixing the extracts with one another at a mixing ratio of 150-250 parts by weight of the Kalopanacis Cortex extract: 650-750 parts by weight of the Chaenomelis Fructus extract: 650-750 parts by weight of the Angelicae Gigantis extract: 50-150 parts by weight of the Zingiberis Rhizoma Crudus extract: 50-150 parts by weight of the Raphani Semen extract: 150-250 parts by weight of the Saururi Herba extract; or
a dry extract mixture obtained by drying each of the extracts and mixing the dried extracts with one another at the above mixing ratio; or a mixed extract obtained by mixing Kalopanacis Cortex, Chaenomelis Fructus, Angelicae Gigantis, Zingiberis Rhizoma Crudus, Raphani Semen and Saururi Herba with one another at the above mixing ratio to prepare a mixture, and extracting the mixture.

10. The composition of claim 9, which has effects on inhibition of inflammatory cytokines and matrix metalloproteinases (MMPs), relief of inflammation and pain, enhancement of activity of cartilage formation factors, and normalization of a joint structure.

11. The composition of claim 9, wherein a number of times the extraction is performed when each of the extracts or mixed extract is prepared is two or more.
